(19) United States
(12) Patent Application Publication
(10) Pub. No.: US 2026/0076798 A1
(43) Pub. Date: Mar. 19, 2026

(54) DEVICE AND METHOD FOR REPLACING A MALFORMED TRICUSPID VALVE

(71) Applicant: Edwards Lifesciences Corporation, Irvine, CA (US)

(72) Inventors: Madison Paige Kukura, Costa Mesa, CA (US); Olivia Marie Conroy, New Rochelle, NY (US); Shruthi Naresh Rajan, Newport Beach, CA (US)

(21) Appl. No.: 19/402,159

(22) Filed: Nov. 26, 2025

Related U.S. Application Data
(63) Continuation of application No. PCT/US2024/031747, filed on May 30, 2024.
(60) Provisional application No. 63/469,956, filed on May 31, 2023.

(51) Int. Cl. A61F 2/24; A61F 2/848

(52) U.S. Cl. CPC A61F 2/2418; A61F 2/2427; A61F 2002/8483; A61F 2210/0014; A61F 2210/0057; A61F 2210/0076; A61F 2230/0069

(57) ABSTRACT

A system for improving the function of a defective heart includes a tubular membrane, a replacement valve, a set of one or more inlets, and a set of one or more outlets. The tubular membrane encompasses (i) a first chamber of the system, shaped to conform to an interior region of a right atrium of the heart, and (ii) a second chamber of the system, shaped to conform to an interior region of a right ventricle of the heart. The replacement valve is configured to separate the first chamber from the second chamber, and has leaflets for providing flow in only one direction. The one or more inlets connect the first chamber with a first portion of cardiovascular anatomy, and the one or more outlets connect the second chamber with a second portion of cardiovascular anatomy.

### CROSS-REFERENCES TO RELATED APPLICATIONS

This application is a Continuation of International Patent Application PCT/US2024/031747 to Kukura et al., filed 30 May 2024, which published as WO 2024/249678, and which claims the benefit of Provisional U.S. Patent Application 63/469,956, filed 31 May 2023, which is hereby incorporated by reference.

### BACKGROUND

The native heart valves (i.e., the aortic, pulmonary, tricuspid and mitral valves) serve critical functions in assuring the forward flow of an adequate supply of blood through the cardiovascular system. These heart valves can be rendered less effective by structural abnormalities congenital malformations, inflammatory processes, infectious conditions, disease, and functional etiologies. Such damage to the valves can result in serious cardiovascular compromise or death. Treatment for such disorders include surgical repair or replacement of the valve during open heart surgery or transvascular techniques for introducing and implanting prosthetic devices in a manner that is much less invasive than open heart surgery.

A healthy heart has a generally conical shape that tapers to a lower apex. The heart has four chambers: the left atrium, right atrium, left ventricle, and right ventricle. The left and right sides of the heart arc separated by a wall generally referred to as the septum. The native tricuspid valve and mitral valve of the human heart connects the right atrium to the right ventricle and left atrium to the left ventricle, respectively. Each valve includes an annulus portion, which is a fibrous structure providing structural support for the valve, and leaflets (also referred to as cusps) that extend downward from the annulus into the ventricle.

When operating properly, the tricuspid valve leaflets function together as a one-way valve to allow blood to flow only from the right atrium to the right ventricle. The right atrium receives deoxygenated blood from the superior and inferior vena cava. When the muscles of the right atrium contract and the right ventricle dilates, the deoxygenated blood that is collected in the right atrium flows into the right ventricle. When the muscles of the right atrium relax and the muscles of the right ventricle contract, the increased blood pressure in the right ventricle urges the leaflets together, thereby closing the one-way tricuspid valve so that blood cannot flow back to the right atrium and is instead expelled out of the right ventricle through the pulmonary valve. To prevent leaflets from prolapsing or flailing under pressure and folding back through the tricuspid annulus toward the right atrium, a plurality of fibrous cords called chordae tendineae tether the leaflets to papillary muscles.

One cardiovascular disorder that can disrupt proper blood flow through the right side of the heart is Ebstein's anomaly (or Ebstein anomaly), which is a congenital heart malformation of the right ventricle and tricuspid valve. During fetal development, the tricuspid valve of these patients fails to fully delaminate from the ventricular myocardium at the atrioventricular junction. As a result, often the septal and posterior leaflets of the tricuspid valve are located toward the apex of the right ventricle and adhered to the myocardium while the anterior leaflet may have a normal attachment to the tricuspid valve annulus at the atrioventricular junction. The degree of displacement of the leaflets, leaflet tethering, and dilation of annulus determine the degree of valve regurgitation, or backward flow of blood into the right atrium. The right ventricle is also malformed and has a thin walled “atrialized” component (the segment of the right ventricle from the level of the “true” tricuspid annulus to that of the displaced septal and posterior leaflets) that balloons out during atrial systole, which may significantly reduce forward flow into the ventricle. During ventricular systole, the ballooned-out portion of the right ventricle contracts, pushing blood backward into the right atrium, even in the absence of significant tricuspid valve regurgitation, resulting in right atrial dilatation. The thick-walled, functional component of the right ventricle is often obstructed by chordal attachments of the anterior leaflet of the tricuspid valve and is smaller than a normal right ventricle. Additionally, an atrial septal defect is present in about 90% of cases. Clinical presentation of symptoms can occur at any age and severe cases can result in heart failure, sudden cardiac arrest, or stroke. Typical treatment is limited in infants and neonate and includes surgical reconstruction of the tricuspid valve, resection of the atrialized ventricle, closure of the atrial septal defect, arrythmia surgery, and right reduction atrioplasty in young children and adults.

### SUMMARY OF THE INVENTION

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In one preferred embodiment, a conduit system for improving the function of a defective heart is provided. The conduit system includes a replacement valve having leaflets for providing flow in only one direction, a tubular membrane encompassing a first chamber and a second chamber separable by the replacement valve, at least one inlet for connecting the first chamber to the anatomy, and at least one outlet for connecting the second chamber to cardiovascular anatomy.

When the conduit is system is used to treat the right side of a heart, the first chamber replaces the function of a right atrium, and the second chamber replaces the function of a right ventricle. The first and second chambers are preferably shaped to conform to interior regions of the right atrium and right ventricle, respectively.

To facilitate deployment, the tubular membrane and replacement valve may be collapsible for allowing implantation using a transcatheter procedure. The replacement valve may be integral with the tubular membrane or may be delivered separately.

When the replacement valve is delivered separately, a dock may be provided within the membrane, wherein the replacement valve is capable of being secured to the dock.

In some aspects, the membrane may be formed from multiple layers. For example, the membrane may include a structural layer and an impermeable layer.

In some aspects, the membrane includes at least one structural layer disposed between two impermeable layers.

In some aspects, the structural layer includes an expandable wire mesh frame formed from a shape memory material.

In some aspects, the structural layer includes an expandable wire mesh interconnected to a strut frame.

In some aspects, at least one impermeable layer is formed from stretchable material.

In some aspects, each impermeable layer membrane includes folded or crinkled portions.

In some aspects, anchoring mechanisms, such as barbs, hooks or helical screws, are provided along an outer surface of the tubular membrane for attachment to surrounding tissue.

In some aspects, at least one impermeable layer includes an outer layer that includes a thick fuzzy material.

In some aspects, at least one impermeable layer includes an outer layer that has been coated with a reendothelialization-inducing biologic.

In some aspects, at least one impermeable layer includes an inner layer that includes a fluorinated polymer.

In some aspects, the tubular membrane has a shape that conforms to interior regions of a right side of a heart, wherein the first chamber is configured to engage an inner wall of a right atrium, the second chamber is configured to engage an inner wall of a right ventricle, and the replacement valve is provided to replace the function of a native tricuspid valve.

In some aspects, the membrane has a shape that conforms to interior regions of a left side of a heart, wherein the first chamber is configured to secure to an inner wall of a left atrium, the second chamber is configured to secure to an inner wall of a left ventricle, and the replacement valve is configured to function as a replacement of a mitral valve.

In some aspects, portion of the membrane are configured to be secured within the cardiovascular anatomy via sutures.

In some aspects, the membrane is configured to be secured within the cardiovascular anatomy via stents.

In another preferred embodiment, an expandable conduit system for installation via sutures within a recipient's cardiovascular system is provided. The conduit system includes a replacement valve, a membrane encompassing a first chamber and a second chamber separable by the replacement valve, and a set of one or more inlets for connecting the first chamber with cardiovascular anatomy of a recipient. At least one inlet may include a region, such as a seam, for suturing to the cardiovascular anatomy. At least one outlet is provided for connecting the second chamber with cardiovascular anatomy of the recipient, wherein each outlet of the set includes a region, such as a seam, for suturing to the cardiovascular anatomy.

In some aspects, each seam is provided on an edge of an inlet or on and edge of an outlet.

In some aspects, the membrane is configured to be sutured within a right side of a heart, wherein the first chamber is configured to secure to an inner wall of a right atrium, the second chamber is configured to secure to an inner wall of a right ventricle, and the replacement valve replaces the function of a native tricuspid valve.

In some aspects, the set of one or more inlets includes a first inlet configured to be sutured to a superior vena cava and a second inlet configured to be sutured to an inferior vena cava.

In some aspects, the set of one or more inlets includes an inlet configured to be sutured to an inner wall of the right atrium such that the inlet encircles a circumference of the atrium.

In some aspects, the set of one or more outlets includes an outlet configured to be sutured to a right ventricular outflow tract.

In some aspects, the set of one or more outlets includes an outlet configured to be sutured to an inner wall of the right ventricle such that the outlet encircles a circumference of the ventricle.

In some aspects, the membrane is configured to be sutured within a left side of a heart, wherein the first chamber is configured to be secured to an inner wall of a left atrium, the second chamber is configured to be secured to an inner wall of a left ventricle, and the replacement valve is configured to function as a replacement of a mitral valve.

In some aspects, one or more inlets are shaped for attachment to native pulmonary veins.

In some aspects, the set of one or more inlets includes an inlet configured to be sutured to an inner wall of the left atrium such that the inlet encircles a circumference of the atrium.

In some aspects, the set of one or more outlets includes an outlet configured to be sutured to a left ventricular outflow tract.

In some aspects, the set of one or more outlets includes an outlet configured to be sutured to an inner wall of the left ventricle such that the outlet encircles a circumference of the ventricle.

In some aspects, the membrane includes a structural layer and at least one impermeable layer.

In some aspects, the structural layer is disposed between two impermeable layers.

In some aspects, the structural layer includes an expandable wire mesh frame formed of a material with shape memory.

In some aspects, the structural layer includes an expandable interconnected to a strut frame formed of a material with shape memory.

In some aspects, each impermeable layer includes a material that is stretchable.

In some aspects, each impermeable membrane includes crinkled portions.

In some aspects, the membrane includes barbs, hooks, or helical screws for securement into native tissue.

In some aspects, the replacement valve includes a set of leaflets capable of opening to form an aperture and coapting to close the aperture. The leaflets are preferably made from pericardium, such as bovine or porcine pericardium. However, the leaflets may also be formed from a synthetic material, such as a polymer or film. In certain embodiments, the leaflets may be formed from the same material as the tubular membrane.

In some aspects, the replacement valve is preloaded and secured within the membrane, such that the first chamber and second chamber are separated.

In some aspects, the system further includes a dock that is secured within the membrane, wherein the replacement valve is capable of docking into the dock such that the first chamber and second chamber are separated.

In another preferred embodiment, the techniques described herein relate to an expandable conduit system for installation via sutures within a recipient's cardiovascular system. The conduit system includes a replacement valve, a membrane encompassing a first chamber and a second chamber separable by the replacement valve, a set of one or more inlets for connecting the first chamber with cardiovascular anatomy of a recipient, wherein each inlet of the set includes an expandable stent for securing to the cardiovascular anatomy. A set of one or more outlets arc provided for connecting the second chamber with cardiovascular anatomy of the recipient, wherein each outlet of the set includes an expandable stent for securing to the cardiovascular anatomy.

In some aspects, each expandable stent is configured to be secured within the cardiovascular anatomy using a transcatheter technique.

In some aspects, each expandable stent is self-expanding.

In some aspects, each expandable stent is configured to be expanded by an inflatable balloon.

In some aspects, the membrane is configured to be secured via stents within a right side of a heart, wherein the first chamber is configured to be secured to an inner wall of a right atrium, the second chamber is configured to be secured to an inner wall of a right ventricle, and the replacement valve is configured to function as a replacement of a tricuspid valve.

In some aspects, the set of one or more inlets includes a first inlet configured to be secured via the expandable stent within a superior vena cava and a second inlet is configured to be secured via the expandable stent within an inferior vena cava.

In some aspects, the set of one or more inlets includes an inlet configured to be secured via the expandable stent within an inner wall of the right atrium such that the inlet encircles a circumference of the atrium.

In some aspects, the set of one or more outlets includes an outlet configured to be secured via the expandable stent within a right ventricular outflow tract.

In some aspects, the set of one or more outlets includes an outlet of the conduit is configured to be secured via a stent within an inner wall of the right ventricle such that the outlet encircles a circumference of the ventricle.

In some aspects, the membrane is configured to be secured via stents within a left side of a heart, wherein the first chamber is configured to be secured to an inner wall of a left atrium, the second chamber is configured to be secured to an inner wall of a left ventricle, and the replacement valve is configured to function as a replacement of a mitral valve.

In some aspects, the inlets are configured to be secured via the expandable stent to native pulmonary veins, such as the left upper pulmonary vein and the left lower pulmonary vein.

In some aspects, the set of one or more inlets includes an inlet configured to be secured via the expandable stent within an inner wall of the left atrium such that the inlet encircles a circumference of the atrium.

In some aspects, the set of one or more outlets includes an outlet configured to be secured via the expandable stent within a left ventricular outflow tract.

In some aspects, the set of one or more outlets includes an outlet configured to be secured via the expandable stent within an inner wall of the left ventricle such that the outlet encircles a circumference of the ventricle.

In some aspects, the membrane includes a structural layer and at least one impermeable layer.

In some aspects, the membrane includes a structural layer sandwiched in between two impermeable layers.

In some aspects, the structural layer includes an expandable wire mesh frame formed of a material with shape memory.

In some aspects, the structural layer includes an expandable interconnected to a strut frame formed of a material with shape memory.

In some aspects, each impermeable layer includes a material that is stretchable.

In some aspects, each impermeable membrane includes folded or crinkled portions.

In some aspects, the membrane includes barbs, hooks, or helical screws, preferably disposed along, or extending from, an exterior surface of the membrane for attachment to surrounding tissue.

In some aspects, the replacement valve includes a set of leaflets capable of opening to form an aperture and coapting to close the aperture.

In some aspects, the replacement valve is preloaded and secured within the membrane, such that the first chamber and second chamber are separated by the replacement valve.

In some aspects, the system further includes a dock that is secured within the membrane, wherein the replacement valve is capable of attachment to the dock after the membrane has been deployed.

In another aspect of the invention, a method for treating a malformed tricuspid valve includes delivering a set of implants to a right atrium of a heart, wherein the set of implants includes a stent, a first replacement valve, and second replacement valve. The stent is expanded within the malformed tricuspid valve. The first replacement valve is expanded within a superior vena cava. The second replacement valve is expanded within an inferior vena cava.

In some aspects, the set of implants further includes a plug, the method further including expanding the plug within an atrial septal defect.

In some aspects, the set of implants are delivered to the right atrium by advancing a delivery catheter through the inferior vena cava.

In some aspects, the set of implants are delivered to the right atrium by advancing a delivery catheter through a femoral vein.

In some aspects, the set of implants are delivered to the right atrium by advancing a delivery catheter through the superior vena cava.

In some aspects, a catheter loaded with the set of implants is delivered to the right atrium via a transjugular approach.

In another aspect of the invention, the techniques described herein are well suited for treating a patient suffering from Ebstein's anomaly. The treatment system may include a flexible hollow structure for deployment in the right side of a heart, wherein the hollow structure is configured to generally conform to interior regions of an atrium and a ventricle, wherein blood passes through the hollow structure from an inlet end to an outlet end and wherein the hollow structure includes a one-way valve for replacing the function of an atrioventricular native valve.

In some aspects, the system further includes anchors for securing the inlet and outlet ends of the hollow structure to the heart.

In some aspects, the hollow structure provides a replacement conduit that extends from the inferior and superior vena cava to the right ventricular outflow tract, thereby effectively replacing the function of the right side of the heart.

In some aspects, the hollow structure is adapted to change shape for conforming to the heart while the heart is beating.

In some aspects, the hollow structure is configured to be implanted using a transcatheter approach.

In some aspects, the anchors include stents for securing the inlet and outlet ends of the hollow structure to the native heart.

In some aspects, the inlet end is anchored to both the inferior vena cava and superior vena cava and the outlet end is anchored to the right ventricular outflow tract.

In some aspects, the hollow structure is reinforced for enhanced rigidity.

In some aspects, the hollow structure is reinforced by a metallic mesh structure.

In some aspects, the hollow structure is flexible and stretchable for replacing the function of one side of the heart.

In some aspects, the hollow structure has impermeable walls.

In some aspects, the hollow structure is adapted to be attached to an inner wall of the heart.

In some aspects, the hollow structure is shaped to configure to the right side of a heart.

Any of the above systems, devices, apparatuses, components, etc. can be sterilized (e.g., with heat, radiation, ethylene oxide, hydrogen peroxide, etc.) to ensure they are safe for use with patients, and the above methods can comprise (or additional methods consist of) sterilization of one or more systems, devices, apparatuses, components, etc. herein (e.g., with heat, radiation, ethylene oxide, hydrogen peroxide, etc.).

### BRIEF DESCRIPTION OF THE DRAWINGS

The description and claims will be more fully understood with reference to the following figures, which are presented as examples of the disclosure and should not be construed as a complete recitation of the scope of the disclosure.

FIG. 1 provides a view of a healthy heart.

FIG. 2 provides a view of an example of a heart affected by Ebstein's anomaly.

FIGS. 3A to 3C provide views of an example of a right-heart bag prosthetic.

FIG. 3D provides a view of an example of a replacement heart valve for use within a bag prosthetic.

FIG. 3E provides a cross-sectional view of the example of a right-heart bag prosthetic.

FIG. 4 provides a view of an example of a right-heart bag prosthetic installed within the heart.

FIGS. 5A and 5B provide views of an example of a right-heart bag prosthetic incorporating stents.

FIG. 5C provides a view of an example of a replacement heart valve for use within a bag prosthetic.

FIG. 6 provides a view of an example of a right-heart bag prosthetic incorporating stents installed within the heart.

FIGS. 7A to 7E provide views of an example of a system and method for treatment of Ebstein's anomaly using a set of implants.

### DETAILED DESCRIPTION OF EMBODIMENTS

The current disclosure describes systems, devices and methods for treating Ebstein's anomaly or other disorders affecting the morphology of the heart chambers and valves. The systems and devices can utilize a set of components that can be provided as a solution to treating an entire side of a heart. In some implementations, the set of components are provided as an all-in-one solution. In some implementations, the set of components can be installed in a set of steps. In some implementations, the systems and devices can be delivered via a transcatheter delivery system. Accordingly, systems and devices can comprise a catheter that provides a means for delivering prosthetics for repairing a side of the heart by installing the prosthetics therein. The systems, devices, and methods described herein can be utilized as an independent treatment or utilized in a combination with any other compatible treatment for repair of cardiac tissue.

Some of the systems and devices of the current disclosure are directed to repairing a side of the heart via implantation of a flexible hollow structure, such as, in the form of a three-dimensional membrane, that can replace the interior regions of an atrium, a ventricle, and the function of an atrioventricular valve therebetween. These systems and devices preferably include one-way valve mechanisms for replacing the function of a tricuspid valve or mitral valve. In some implementations, the systems and devices comprise a means for attachment, which can be performed by a surgical procedure. In some implementations, the systems and devices comprise a set of expandable stents or other anchoring mechanisms for securing inlet and outlet ends of the device. The hollow structure provides a replacement conduit for ensuring that blood efficiently passes through the heart in the proper direction. The hollow structure is flexible and/or pliable such that when installed and secured, the membrane contracts and expands by the forces of the heart muscles as the heart is beating. Implantation is preferably performed by a minimally or non-invasive procedure utilizing a transcatheter system; however, implantation may also be performed using a surgical procedure.

Some of the systems and devices of the current disclosure are directed to repairing a side of the heart via a set of valves, stents, and plugs. These systems and devices can be utilized to replace a tricuspid valve or mitral valve and occlude atrial septal defects. In some implementations, the systems and devices comprise a set of expandable valves, an expandable stent, and an expandable plug such that they can installed via a minimally or non-invasive procedure utilizing a transcatheter system.

The described systems, devices, and methods should not be construed as limiting in any way. Instead, the present disclosure is directed toward all novel and nonobvious features and aspects of the various disclosed systems and devices, alone and in various combinations and subcombinations with one another. The disclosed systems, devices, and methods are not limited to any specific aspect, feature, or combination thereof, nor do the disclosed systems, devices, and methods require that any one or more specific advantages be present, or problems be solved.

Various examples of systems and components for repair of heart disorders are disclosed herein, and any combination of these examples can be made unless specifically excluded. For example, a means for attachment utilized within some embodiments can be combined with a set of one or more expandable stents within other embodiments, even if a specific combination is not explicitly described. Likewise, the different constructions and features of systems for repair can be mixed and matched, such as by combining the various components of the systems, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

The terms “proximal” and “distal” as used throughout the description relate to a catheter system axis, in which the end where the procedure is performed is the distal end and the opposite end where the catheter system is controlled is the proximal end. Accordingly, the distal end of the catheter system is the leading end that first traverses into the body and first reaches the procedure site. Conversely, the proximal end of the catheter system is the portion that remains extracorporeal. Likewise, a distal movement along the catheter axis would be movement of a component in a direction towards (or beyond) a site of procedure and a proximal movement along the catheter axis would be movement of a component in an opposite direction. Although these terms have a relationship with a site of procedure, it is to be understood that these terms are used for reference and the site of procedure does not need to be present when interpreting the components or movements of the devices and systems described herein.

Various systems and devices for repair are utilized for the purpose of performing a procedure within a recipient. Recipients include (but are not limited to) patients, animal models, cadavers, or anthropomorphic phantoms. Accordingly, in addition to methods of treating patients, the systems and devices can be utilized in training or other practice procedures upon animal models, cadavers, or anthropomorphic phantoms. Further, the techniques, methods, operations, steps, etc. described or suggested herein can be performed on a living animal or on a non-living simulation, such as on a cadaver, cadaver heart, anthropomorphic ghost, simulator (e.g., with the body parts, tissue, etc. being simulated), etc.

The described systems and devices can be sterilized, which can be performed using gamma irradiation, gas plasma, aldehydes, ethylene oxide, and/or e-beam. The systems or devices can be further treated with a formaldehyde bioburden reduction process. After preparation, the systems and devices can be stored within a container, which can be hermetically sealed or otherwise kept sterile.

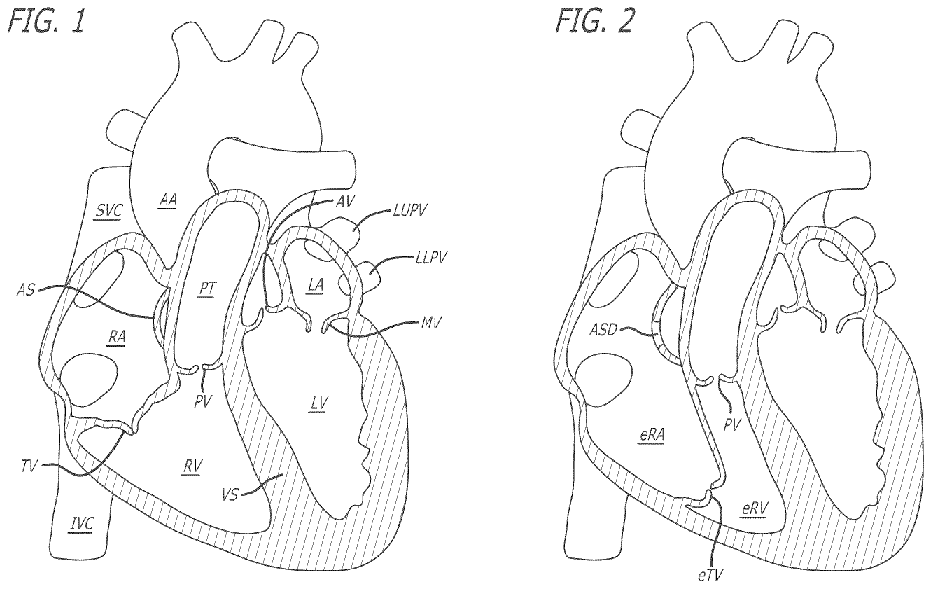

FIG. 1 is a coronal-plane view of the heart sectioning through the heart such that the four chambers and the four valves of the heart can be viewed. The right ventricle (RV) and the right atrium (RA) are separated from the left ventricle (LV) and the left atrium (LA) via the ventricular septum (VS) and atrial septum (AS), respectively. The four heart valves direct flow between the four chambers. The RA is separated from the RV via the tricuspid valve (TV). The blood from the RV is pumped through the pulmonary valve (PV) into the pulmonary trunk (PT) and lungs. The LA is separated from the LV via the mitral valve (MV). The blood from the LV is pumped through the aortic valve (AV) into the aortic arch (AA) and throughout the body.

When operating properly, the leaflets of each valve function together as a one-way valve to allow blood to flow only in one direction. The right atrium receives unoxygenated blood returning from the peripheral vascular system via the superior vena cava (SVC) and inferior vena cava (IVC). When the muscles of the RA contract and the RV dilates, the deoxygenated blood that is collected in the RA flows into the RV via the TV. When the muscles of the RA relax and the muscles of the RV contract, the increased blood pressure in the RV urges the leaflets of the TV to coapt together, thereby closing the valve so that blood cannot flow back to the RA and is instead expelled out of the RV through the PV into the lung capillaries where the hemoglobin of the red blood cells are oxygenated.

The LA receives oxygenated blood from the pulmonary veins (LUPV and LLPV). When the muscles of the LA contract and the LV dilates, the oxygenated blood that is collected in the LA flows into the LV via the MV. When the muscles of the LA relax and the muscles of the LV contract, the increased blood pressure in the LV urges the two leaflets of the mitral valve to coapt together, thereby closing the valve so that blood cannot flow back to the LA and is instead expelled out of the LV through the AV.

FIG. 2 provides a coronal-plane view of an example of a heart affected by Ebstein's anomaly. The tricuspid valve (eTV) is much lower, resulting in a greatly expanded right atrium (eRA) and greatly reduced right ventricle (eRV). The leaflets of the eTV are malformed, resulting in retrograde regurgitation. The atrial septum may also have a hole referred to as an atrial-septal defect (ASD), which allows blood to transfer from atrium into the other. The pulmonary valve (PV), and the rest of the heart have fairly typical morphology and thus correction of the right side of the heart could yield great benefit for patients afflicted with Ebstein's anomaly.

Systems and devices for repair comprise a conduit in the form of a three-dimensional membrane having the shape of the right chambers or of the left chambers of the heart, such that it can act as surrogate of those chambers. The systems and devices can comprise a means for attachment and/or securement within the heart. The system and devices further comprise a prosthetic replacement valve within the membrane.

Some implementations of the systems and devices for repair can be utilized on the right side, while other implementations of the systems and devices for repair can be utilized the left side of the heart. The various systems and devices can incorporate a replacement tricuspid valve or a replacement mitral valve, respective to the side of heart being repaired. Any surgical approach can be utilized to reach the internal chambers, such as (for example) open heart surgery. Alternatively, a minimally or non-invasive transcatheter approach may be used. When a transcatheter approach is utilized, the internal chambers can be reached via a transfemoral, transjugular, subclavian, transapical, or transaortic approach.

In some implementations, the conduit is shaped such that it conforms to at least a portion of an atrium and ventricle. In some implementations, the conduit encompasses a set of internal chambers. In some implementations, the conduit comprises a set of two chambers separated by a replacement valve. In some implementations, the conduit comprising a set of two internal chambers separated by a replacement valve further comprises one or more inlet and/or an outlet for connecting with cardiovascular anatomy of a recipient. In some implementations, the conduit provides unidirectional blood from an atrium into a ventricle through an internal replacement valve.

In some implementations, the conduit comprises a replacement valve capable of providing unidirectional blood flow. The replacement valve can be preloaded within the conduit, or it can be delivered to the conduit by a transcatheter technique. In some implementations, a preloaded replacement valve is attached to the conduit, which can be achieved using sutures, staples, hooks, interlocking mechanism, or any other means for securing a valve therein. In some implementations, a docking station is attached to the conduit, the docking station comprises a means for installing a replacement valve therein. Regardless of the means of securement of the replacement valve within the conduit, the securement should provide a seal such that there is no perivalvular leakage within the membrane, ensuring unidirectional blood flow.

In some implementations, the replacement valve is expandable. In some implementations, an inflatable balloon can be utilized to expand a replacement valve. In some implementations, the replacement valve is a self-expanding, which can be achieved using a shape-memory material like nitinol. In some implementations, an annular portion of the replacement valve comprises a number of overlapping segments, which can allow for expansion of a replacement valve. In some implementations, when unexpanded, the replacement valve and conduit is capable of fitting within a catheter such that it can be delivered to the site of installation via a transcatheter technique.

A replacement valve can be a leaflet-based valve or a mechanical valve. In some implementations, a replacement valve comprises a set of leaflets capable of opening to form an aperture and coapting to close the aperture. Accordingly, the set of leaflets can open and close in accordance with the systolic and diastolic beats of the heart. Any appropriate number of leaflets can be utilized, typically two or three leaflets such that the set of leaflets mimic the native valve to be replaced, but can be up to four, five, or 6 leaflets. The leaflet material can be a biocompatible polymer, animal tissue (e.g., pericardium), synthetic tissue, fabric, or film. Each leaflet can comprise a cusp portion that is in connection with a valve frame or directly in connection with a portion of the inner face of the conduit. Each can comprise a free edge that is capable of coapting with one or more free edges of another leaflet such that the set of leaflet free edges close the valve. In some embodiments, a mechanical valve is utilized, capable of opening to form an aperture and coapting to close the aperture.

In some implementations, at least a portion of the valve comprises a cover. In some implementations, a frame of a leaflet-based valve comprises a cover. In some implementations, a mechanical valve comprises a cover. The cover can be a biocompatible polymer, animal tissue, or synthetic tissue. In some implementations, the cover allows for expansion of the valve. Accordingly, a cover can comprise a stretchable material and/or can provide slack (e.g., one or more folded or crinkled portions) such that the valve can expand during installation and/or as a recipient's heart grows.

In some implementations, the conduit conforms to interior regions of the right side of the heart and comprises an inner right atrium and an inner right ventricle separated by a replacement tricuspid valve. In some implementations, the conduit conforming to interior regions of the right side of the heart further comprises an inlet for connection with a superior vena cava, and/or an inlet for connection with an inferior vena cava, and/or an outlet for connection with a right ventricular outflow tract outlet.

In some implementations, the conduit conforms to interior regions of the left side of the heart and comprises an inner left atrium and an inner left ventricle separated by a replacement mitral valve. In some implementations, the conduit conforming to interior regions of the left side of the heart further comprises an inlet for connection with a left upper pulmonary vein, and/or an inlet for connection with a left lower pulmonary vein, and/or an outlet for connection with a left ventricular outflow tract outlet.

In some implementations, the membrane comprises a plurality of layers. In some implementations, the membrane comprises at least one structural layer and at least one impermeable layer. In some implementations, the conduit comprises at least one structural layer sandwiched in between two impermeable layers.

In some implementations, a conduit formed by a three-dimensional membrane is expandable. An expandable three-dimensional membrane can provide various benefits. One benefit of expandability is that it can facilitate installation by expanding to the inner walls within the heart chambers. Another benefit is when an expandable conduit is utilized to repair a juvenile heart, the membrane can expand as the heart grows. In some implementations, a structural layer comprises an expandable wire mesh frame or an expandable interconnected to a strut frame, which can be comprise a material with shape memory (e.g., nitinol). In some implementations, a conduit formed by a three-dimensional membrane is flexible and/or pliable, which can allow the membrane to expand and contract with the chambers of the heart as it beats. In some implementations, an impermeable layer comprises a material that is stretchable. Impermeable materials that are stretchable include (but are not limited to) poly(ethylene) (PE), poly(ethylene terephthalate) (PET), ultra-high molecular weight poly(ethylene) (UHMWPE), thermoplastic poly(urethane) (TPU), expanded poly (tetrafluoroethylene) (ePTFE), and Dyneema; which can be combined and/or fluorinated. In some implementations, the impermeable membrane comprises crinkled portions such that when the membrane expands, the crinkles flatten out (like an accordion), allowing for expansion.

In some implementations, the external face of the three-dimensional membrane is configured to interact with the native cardiac tissue, which can improve securement, reduce leakage around the membrane, promote tissue ingrowth and/or promote healing. In some implementations, the external face of the three-dimensional membrane comprises barbs, hooks and/or helical screws for securement into the native tissue. In some implementations, the external face of the three-dimensional membrane comprises a thick fuzzy material (e.g., thicker than the impermeable layer), which can promote tissue ingrowth and prevent leakage around the membrane. In some implementations, the external face of the three-dimensional membrane has been coated with a reendothelialization-inducing biologic, such as (for example) amino acids (e.g., lysine or ornithine), saccharides (e.g., hyaluronic acid, fibronectin, chitosan), structural proteins (e.g., collagen, elastin), growth factors (e.g., VEGF), or a combinations thereof.

In some implementations, the conduit is adapted to be attached to an inner surface of the heart. In some implementations, the conduit is configured to be secured within the heart via sutures. In some implementations, the inlets and the outlets of the conduit are configured to be securely sutured to the cardiovascular anatomy. In some implementations, the inlets and the outlets each comprise a scam for suturing to the cardiovascular anatomy. In some implementations, a seam is provided on the edge of an inlet or an outlet.

In some implementations, the conduit is configured to be sutured within the right side of the heart. In some implementations, a first inlet of the conduit is configured to be sutured to the superior vena cava and a second inlet of the conduit is configured to be sutured to the inferior vena cava. In some implementations, an inlet of the conduit is configured to be sutured to an inner wall of the right atrium such that the inlet encircles a circumference of the atrium. In some implementations, an outlet of the conduit is configured to be sutured to the right ventricular outflow tract. In some implementations, an outlet of the conduit is configured to be sutured to an inner wall of the right ventricle such that the outlet encircles a circumference of the ventricle.

In some implementations, the conduit is configured to be sutured within the left side of the heart. In some implementations, a first inlet of the conduit is configured to be sutured to the left upper pulmonary vein and a second inlet of the conduit is configured to be sutured to the left lower pulmonary vein. In some implementations, an inlet of the conduit is configured to be sutured to an inner wall of the left atrium such that the inlet encircles a circumference of the atrium. In some implementations, an outlet of the conduit is configured to be sutured to the left ventricular outflow tract. In some implementations, an outlet of the conduit is configured to be sutured to an inner wall of the left ventricle such that the outlet encircles a circumference of the ventricle.

In some implementations, the conduit is configured to be secured within the heart via stents. In some implementations, the inlets and the outlets of the conduit are configured to be secured to the cardiovascular anatomy via stents. In some implementations, stents are configured to be secured within the cardiovascular anatomy using a transcatheter technique. In some implementations, the stents are self-expanding. In some implementations, the stents are configured to be expanded by an inflatable balloon.

In some implementations, the conduit is configured to be secured via stents within the right side of the heart. In some implementations, a first inlet of the conduit is configured to be secured via a stent within the superior vena cava and a second inlet of the conduit is configured to be secured via a stent within the inferior vena cava. In some implementations, an inlet of the conduit is configured to be secured via a stent within an inner wall of the right atrium such that the inlet encircles a circumference of the atrium. In some implementations, an outlet of the conduit is configured to be secured via a stent within the right ventricular outflow tract. In some implementations, an outlet of the conduit is configured to be secured via a stent within an inner wall of the right ventricle such that the outlet encircles a circumference of the ventricle.

In some implementations, the conduit is configured to be secured via stents within the left side of the heart. In some implementations, a first inlet of the conduit is configured to be secured via a stent within the left upper pulmonary vein and a second inlet of the conduit is configured to be secured via a stent within the left lower pulmonary vein. In some implementations, an inlet of the conduit is configured to be secured via a stent within an inner wall of the left atrium such that the inlet encircles a circumference of the atrium. In some implementations, an outlet of the conduit is configured to be secured via a stent within the left ventricular outflow tract. In some implementations, an outlet of the conduit is configured to be secured via a stent within an inner wall of the left ventricle such that the outlet encircles a circumference of the ventricle.

In some implementations, the conduit is adapted to be attached to inner walls of the heart. Any means of attachment or securement to the inner walls can be utilized. In some implementations, sutures are utilized to attach or secure the conduit to an inner wall. In some implementations, barbs are utilized to attach or secure the conduit to an inner wall. In some implementations, anchors are utilized to attach or secure the conduit to an inner wall.

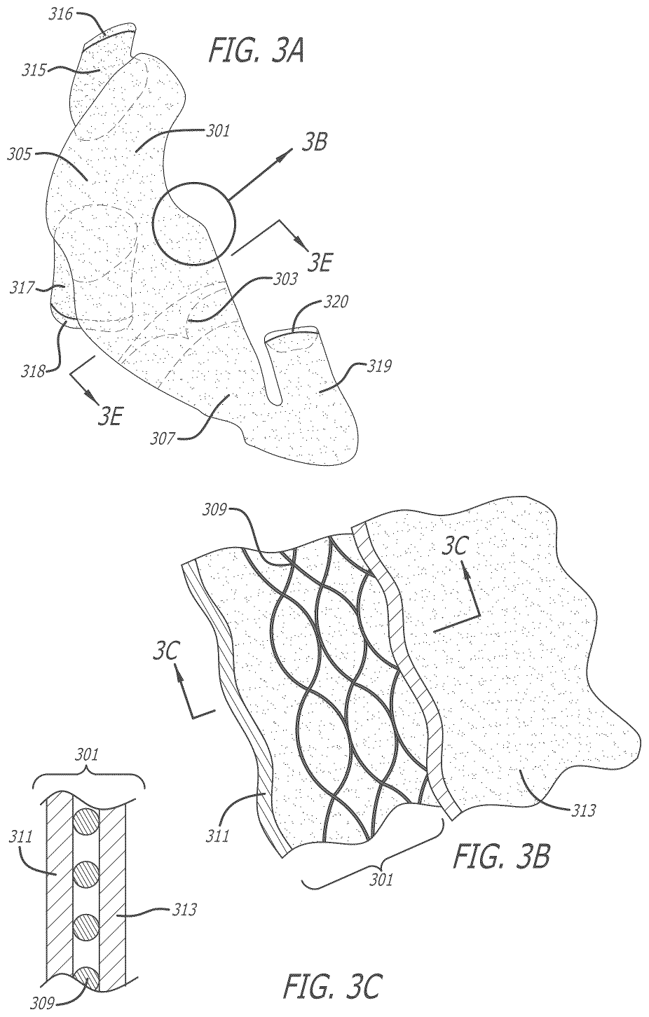

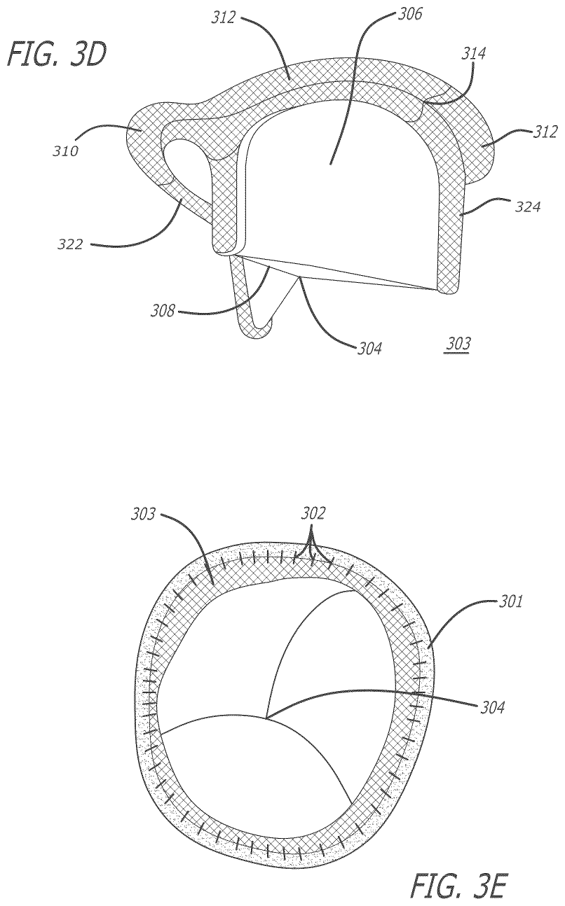

Provided in FIG. 3A is an example of a flexible hollow structure (i.e., conduit) in the form of a three-dimensional membrane that can be implanted into a patient. The system comprises a membrane 301 that is configured to have a three-dimensional shape to conform to interior regions of the right chambers of the heart. The system has a replacement valve 303 within membrane 301, yielding a replacement right atrium chamber 305 above the valve and a replacement right ventricle chamber 307 below the valve.

As shown in FIGS. 3B and 3C, in this example, membrane 301 comprises a structural layer 309 sandwiched between an inner layer 311 and an outer layer 313. Structural layer 309 can comprise a mesh of material that provides an outwardly push force. The material can be a shape-memory material, such as (for example) nitinol that be shaped to conform to interior regions of a right side of a heard and to yield an outwardly push force by sizing the three-dimensional conformation larger than the size of the chambers of a recipient.

FIG. 3D provides one example of a leaflet-based replacement valve 303 that may be used within the conduit system. Replacement valve 303 comprises a set of three leaflets 304. Each leaflet comprises a cusp portion 306 and a free edge 308. Each cusp portion 306 can be attached to a frame 310. The three free edges 308 of the set of leaflets are capable of coming together to close the valve and extending away from the other edges to open the valve, yielding a one-way valve. Frame 310 can comprise a ring base 322 and a set of 2 or more posts 324 (3 posts depicted). Ring base 322 can comprise a plurality of segments 312 that overlap or otherwise allow for expansion, which can overlap at plurality of segment junctions 314. In combination, segments 312 form ring base 322. Posts 324 can be substantially perpendicular to ring base 322 and extend from the ring base. Each post 324 can be secured to one of the segments 312 and provide a means for leaflet 304 attachment. Frame 310 can further comprise a cover.

FIG. 3E provides a view of replacement valve 303 attached within membrane 301. The set of leaflets 304 are depicted closed with the free edges coapted. Ring base 322 of frame 310 can provide an annulus-like area, which is shown to be attached to membrane 301 via a plurality of sutures 302. The interaction between frame 310 and membrane 301 should yield a seal such that there is no perivalvular leakage. Although FIGS. 3A and 3E depict a preloaded replacement valve, it should be understood that a valve can be delivered and installed within the conduit system.

Inner layer 311 and outer layer 313 can each be an impermeable material for preventing blood from passing therethrough. The inner layer and outer layer can also each be a stretchable material such that three-dimensional conformation can expand outward to conform to the interior regions of the right-side chambers of a heart.

When the conduit system is installed within a recipient, inner layer 311 will be in contact with the recipient's blood. The inner layer can be configured to prevent coagulation and/or pannus formation to ensure proper blood flow and prevent clotting. For example, a fluorinated polymer can help prevent coagulation and/or pannus formation.

When the three-dimensional membrane is installed within a recipient, outer layer 313 will be in contact with the inner chamber walls of the recipient. The outer layer can be conformed to promote interaction with the host tissue to promote healthy ingrowth. For instance, the outer layer can comprise a thick fuzzy material (e.g., thicker than the impermeable layer), which can promote tissue ingrowth and prevent leakage around the membrane. The outer layer can also be coated with a reendothelialization-inducing biologic, such as (for example) amino acids (e.g., lysine or ornithine), saccharides (e.g., hyaluronic acid, fibronectin, chitosan), structural proteins (e.g., collagen, elastin), growth factors (e.g., VEGF), and combinations thereof.

As depicted in the example within FIG. 3A, the conduit system comprises a first inlet 315, a second inlet 317, and an outlet 319. First inlet 315 has a shape such that it capable of conforming to the superior vena cava of a recipient and a first inlet seam 316 for attachment to the inner walls of the superior vena cava. Second inlet 317 has a shape such that it capable of conforming to the superior vena cava of a recipient and a second inlet seam 318 for attachment to the inner walls of the inferior vena cava. Outlet 319 has a shape such that it capable of conforming to the right ventricular outflow tract and an outlet seam 320 for attachment to the inner walls of the right ventricular outflow tract. Accordingly, when the conduit system is installed within a recipient, it is configured to be capable of receiving blood from the superior vena cava via first inlet 315 and inferior vena cava via second inlet 317 into replacement right atrium chamber 305. The conduit system is further configured to be capable of transferring blood from replacement right atrium chamber 305 into replacement right ventricle chamber 307 via replacement valve 303, ensuring unidirectional blood flow. From the replacement right ventricle chamber 307, the system is further configured to be capable of transferring blood into the right ventricular outflow tract via outlet 319.

The three-dimension membrane system is configured to be capable of being sutured into and attached to the inner chamber walls of a recipient. Suturing locations can include the first inlet seam 316, the second inlet seam 318, and outlet seam 318 to ensure integration with the blood flow of the cardiovascular system. Suturing locations can also include any other locations along membrane 301 to help attach to the chamber walls. Barbs and/or anchors (e.g., helical screws) can also be incorporated into the membrane to enhance attachment to the walls of the heart chambers.

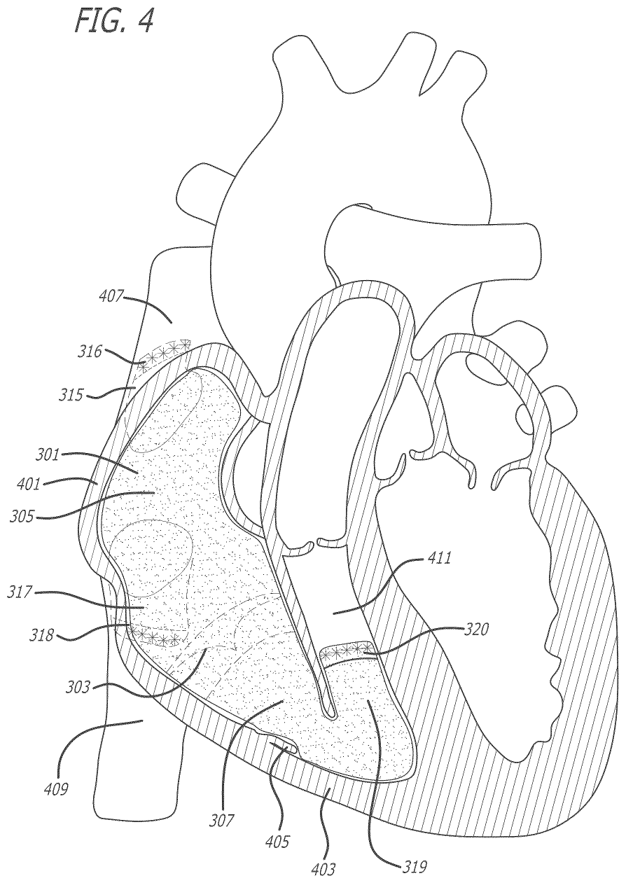

Provided in FIG. 4 is an example of a conduit system installed within a recipient having malformations consistent with Ebstein's anomaly. Membrane 301 is conformed to a shape that contours the walls of the recipient's right atrium 401 and right ventricle 403. The recipient's native tricuspid valve 405, which is malformed and improperly located within the right ventricular area, can be excised and/or pushed to the side during installation of the conduit system. First inlet 315 can conform within the recipient's superior vena cava 407. Second inlet 317 can conform within the recipient's inferior vena cava 409. Outlet 319 can conform within the recipient's right ventricular outflow tract 411.

The conduit system can be secured to the chambers via suturing, barbs, and/or anchors. Specifically, first inlet 315 can be sutured to the superior vena cava 407, second inlet 317 can be sutured to the inferior vena cava 409, and outlet 319 can be sutured to the right ventricular outflow tract 411, ensuring that blood can flow within and through the system. Multiple more attachment points can be utilized to further secure the system to the chamber walls via sutures, barbs, and/or anchors.

With the conduit system secured to the right atrium and ventricle, the system can respond to the contractions and beats of the right heart muscles. Accordingly, the replacement right atrium chamber 305 receives unoxygenated blood returning from the peripheral vascular system via first inlet 315 and second inlet 317. When the muscles of the recipient's right atrium contract and the right ventricle dilates, the deoxygenated blood that is collected in the replacement right atrium chamber 305 flows into the replacement right ventricle chamber 307 via replacement valve 303. When the muscles of the recipient's right atrium relax and the muscles of the recipient's right ventricle contract, the increased blood pressure in the replacement right ventricle chamber 307 urges set of leaflets 304 of replacement valve 303 to coapt together, thereby closing the valve so that blood cannot flow back to the replacement right atrium chamber 305 and is instead expelled out of the replacement right ventricle chamber via outlet 319 and through the pulmonary valve.

While a specific configuration of a conduit system is described above with reference to FIG. 3 A to 4, it should be readily appreciated that various configurations of the system can be implemented in any of a variety of combinations of components. Accordingly, the specific configuration of the system described herein should be understood as not to be limited to any specific configuration, but instead can be implemented in any other appropriate configuration. For example, although the conduit system is described for treatment of a malformed or injured right side of the heart, a similar conduit system can be configured for treatment of a malformed or injured left side of the heart. For example, a system can be utilized on the left side of the heart for patients suffering from heart failure.

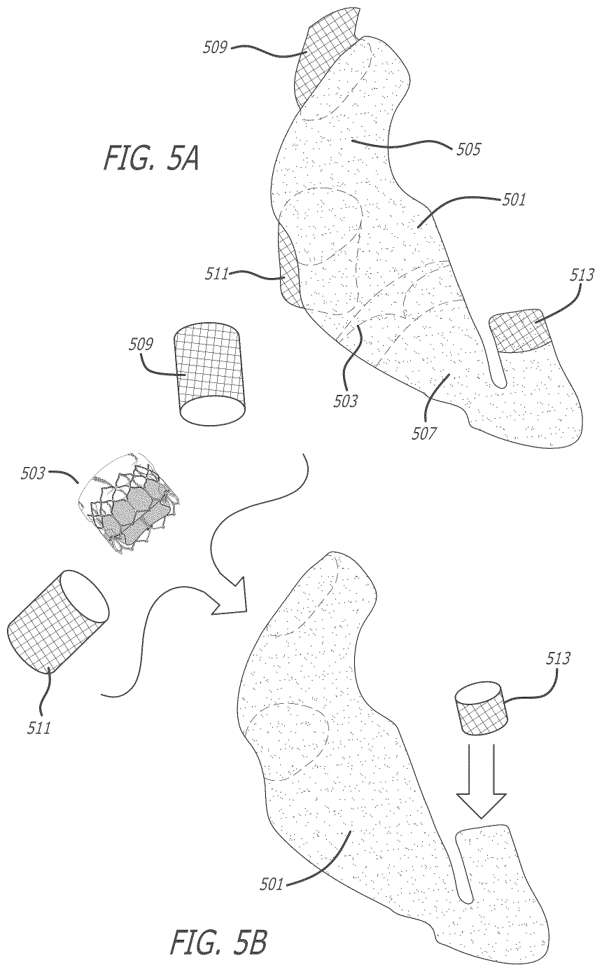

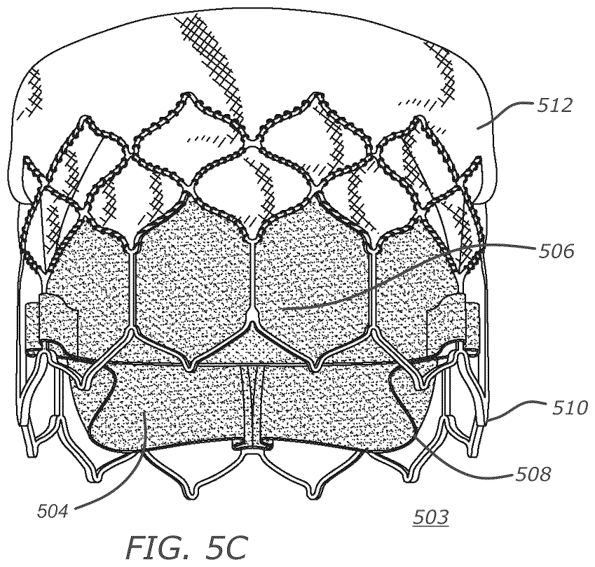

Provided in FIGS. 5A and 5B is an example of a flexible hollow structure (i.e., conduit system) that can be secured within a recipient via a set of stents. The system comprises a membrane 501 that is configured to have a three-dimensional shape to conform to interior regions of the right chamber of the heart. The system has a replacement valve 503 within membrane 501, yielding a replacement right atrium chamber 505 above the valve and a replacement right ventricle chamber 507 below the valve.

Similar to the systems shown within FIGS. 3B and 3C, membrane 501 can comprise a structural layer sandwiched between an inner layer and an outer layer. Structural layer can comprise a mesh of material that provides an outwardly push force. The material can be a shape-memory material, such as (for example) nitinol that be shaped to conform to interior regions of a right side of a heart and to yield an outwardly push force by sizing the three-dimensional conformation larger than the size of the chambers of a recipient.

Replacement valve 503 can be similar-to the valve depicted in FIGS. 3D and 3E, or a replacement valve configured for transcatheter delivery and installation. FIG. 5C provides one example of a leaflet-based replacement valve 503 capable of being delivered via a transcatheter system. Replacement valve comprises a set of three leaflets 504. Each leaflet comprises a cusp portion 506 and a free edge 508. Each cusp portion 506 can be attached to a frame 510. The three free edges 508 of the set of leaflets are capable of coming together to close the valve and extending away from the other edges to open the valve, yielding a one-way valve. Frame 510 can comprise a plurality of interconnected struts that allow the frame to be crimped and elongated along the concentric axis and to be expanded and shortened. The structure of frame 510 can comprise a mechanical means, shape-memory material (e.g., nitinol), or any other means to allow for expansion. Expansion of frame 510 can allow for installation of the replacement valve 503 and/or can allow for the valve to expand post-implantation as the heart grows. Frame 310 can further comprise a cover 512.

The inner layer and the outer layer can each be an impermeable material, which can help ensure that fluid does not flow therethrough. The inner layer and outer layer can also each be a stretchable material such that three-dimensional conformation can expand outward to conform to the right-side chambers of a heart.

When the conduit system is installed within a recipient, the inner layer will be in contact with the recipient's blood. The inner layer can be configured to prevent coagulation and/or pannus formation to ensure proper blood flow and prevent clotting. For example, a fluorinated polymer can help prevent coagulation and/or pannus formation.

When the three-dimensional membrane is installed within a recipient, the outer layer will be in contact with the inner chamber walls of the recipient. The outer layer can be conformed to promote interaction with the host tissue to promote healthy ingrowth. For instance, the outer layer can comprise a thick fuzzy material (e.g., thicker than the impermeable layer), which can promote tissue ingrowth and prevent leakage around the membrane. The outer layer can also be coated with a reendothelialization-inducing biologic, such as (for example) amino acids (e.g., lysine or ornithine), saccharides (e.g., hyaluronic acid, fibronectin, chitosan), structural proteins (e.g., collagen, elastin), growth factors (e.g., VEGF), and combinations thereof.

The conduit system comprises a first stent 509, a second stent 511, and a third stent 513. First stent 509 has a shape such that it capable of expanding within and conforming to the superior vena cava of a recipient. Second stent 511 has a shape such that it capable of expanding within and conforming to the superior vena cava of a recipient. Third stent 513 has a shape such that it capable of expanding within and conforming to the right ventricular outflow tract. Accordingly, when the conduit system is installed within a recipient, it is configured to be capable of receiving blood from the superior vena cava through first stent 509 and inferior vena cava via second stent 511 into replacement right atrium chamber 505. The conduit system is further configured to be capable of transferring blood from replacement right atrium chamber 505 into replacement right ventricle chamber 507 via replacement valve 503, ensuring unidirectional blood flow. From the replacement right ventricle chamber 507, the system is further configured to be capable of transferring blood into the right ventricular′ outflow tract through third stent 513.

The conduit system is configured to be capable of being secured into and attached to the inner chamber walls of a recipient. Securing locations can include the first stent 509, the second stent 511, and third stent 513 to ensure integration with the blood flow of the cardiovascular system. Attachment locations can also include various locations along membrane 301 to help attach to the chamber walls. Sutures, barbs and/or anchors (e.g., helical screws) can also be incorporated into the membrane to enhance attachment to the walls of the heart chambers.

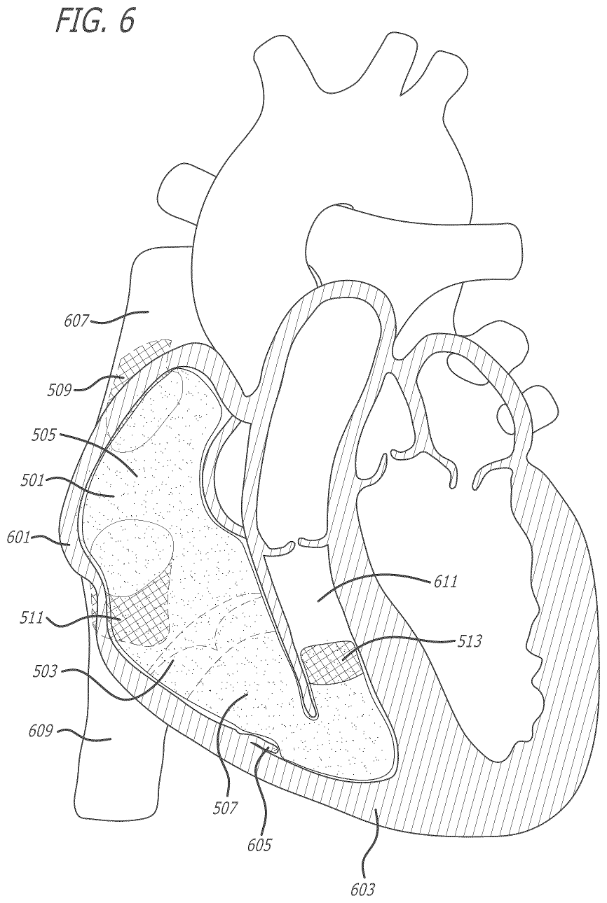

Provided in FIG. 6 is an example of a conduit system installed within a recipient having malformations consistent with Ebstein's anomaly. Membrane 501 is conformed to a shape that contours the walls of the recipient's right atrium 601 and right ventricle 603. The recipient's native tricuspid valve 605, which is malformed and improperly located within the right ventricular area, can be excised and/or pushed to the side during installation of the conduit system. In some implementations, the conduit system is delivered and installed via a transcatheter system. First stent 509 can expand and conform within the recipient's superior vena cava 607. Second stent 511 can expand and conform within the recipient's inferior vena cava 609. Third stent 513 can expand and conform within the recipient's right ventricular outflow tract 611.

The conduit system can be secured to the chambers via the set of stents. Specifically, first stent 509 can provide an outward radial force such that it secures within superior vena cava 607, second stent 511 can provide an outward radial force such that it secures within the inferior vena cava 609, and third stent 513 can provide an outward radial force such that it secures within the right ventricular outflow tract 611, ensuring that blood can flow within and through the system. Each stent can be expanded by a balloon and/or comprise a shape-memory material (e.g., nitinol) such that the stent is configured to expand radially outward. Multiple attachment points can be utilized to further secure the system to the chamber walls via sutures, barbs, and/or anchors.

Replacement valve 503 can be preloaded within the three-dimensional membrane of the conduit systems such that when the conduit system is delivered and installed (e.g., via a transcatheter system), the replacement valve is delivered and installed concurrently. When preloaded, replacement valve 503 can already be secured to the conduit system prior to and during delivery or can be secured to conduit system as part of the installation procedure. Alternatively, replacement valve 503 can be delivered without being preloaded within the conduit system. When replacement valve 503 is not preloaded, it can be delivered concurrently using the same delivery catheter or can be delivered sequentially using a delivery catheter to be delivered subsequent to delivery. In any manner of delivery, and whether preloaded or not, when replacement valve 503 is at the site of installation, it can be expanded within the three-dimensional membrane of the conduit system, and optionally further secured therein. In some implementations, a conduit system further comprises a dock for docking and securing the valve within the three-dimensional membrane of the conduit system.

With the conduit system secured to the right atrium and ventricle, the system can respond to the contractions and beats of the right heart muscles. Accordingly, the replacement right atrium chamber 505 receives unoxygenated blood returning from the peripheral vascular′ system through first stent 509 and second stent 511. When the muscles of the recipient's right atrium contract and the right ventricle dilates, the deoxygenated blood that is collected in the replacement right atrium chamber 505 flows into the replacement right ventricle chamber 507 via replacement valve 503. When the muscles of the recipient's right atrium relax and the muscles of the recipient's right ventricle contract, the increased blood pressure in the replacement right ventricle chamber 507 urges the leaflets of the replacement valve 503 to coapt together, thereby closing the valve so that blood cannot flow back to the replacement right atrium chamber 505 and is instead expelled out of the replacement right ventricle chamber through third stent 513 and the pulmonary valve.

While a specific configuration of a conduit system is described above with reference to FIG. 5A to 6, it should be readily appreciated that various configurations of the system can be implemented in any of a variety of combinations of components. Accordingly, the specific configuration of the system described herein should be understood as not to be limited to any specific configuration, but instead can be implemented in any other appropriate configuration. For example, although the conduit system is described for treatment of a malformed right side of the heart, a similar conduit system can be configured for treatment of a malformed left side of the heart.

Systems and devices for repairing the right side of a heart comprise a transcatheter delivery system such that it can be utilized within minimally or non-invasive procedures and/or traverse through the circulatory system to reach the site of procedure. The systems and devices can comprise one or more delivery catheters for delivery and installing a set of implants inclusive of plugs, stents, and valves. Delivery catheter systems and devices can comprise one or more plugs for repairing malformed holes or injuries within the cardiovascular system. Delivery catheter systems and devices can comprise one or more stents for opening of anatomies that can cause blood flow disruption. Delivery catheter systems and devices can comprise one or more replacement valves for replacement. In some implementation, a single delivery catheter can comprise all the components necessary for repair. In some implementations, a plurality of delivery catheters is utilized to delivery all the components necessary for repair, each of the delivery catheters to be utilized sequentially.

The systems and devices for implant delivery can be utilized malformations of any of the four valves of the heart, including the mitral valve, the tricuspid valve, the pulmonary valve, and the aortic valve. Any transcatheter approach can be utilized to reach the valve to be treated, such as (for example) a transfemoral, transjugular, subclavian, transapical, or transaortic approach. In some implementations, the set of components are used for repair of a heart having malformations consistent with Ebstein's anomaly.

A set of one or more components can be installed. Each component of the set of can be strategically installed at particular locations within the cardiovascular system to replace the anatomical malformations and repair function. In some implementations, a malformed and inappropriately located valve can be opened up with stent such that the valve no longer causes turbulent blood flow. In some implementations, an ineffective valve is replaced with a set of one or two replacement valves. In some implementations, a septal defect is occluded with a plug.

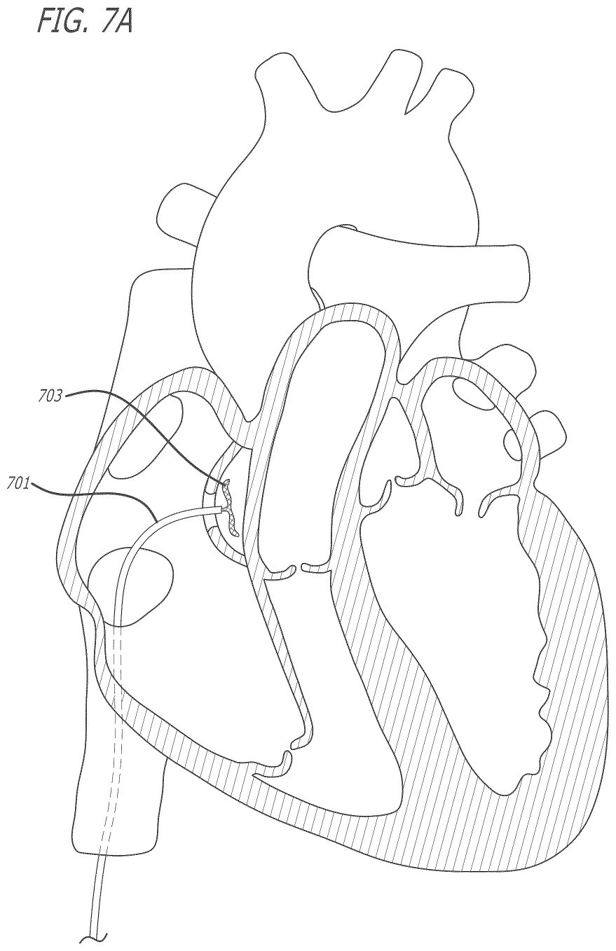

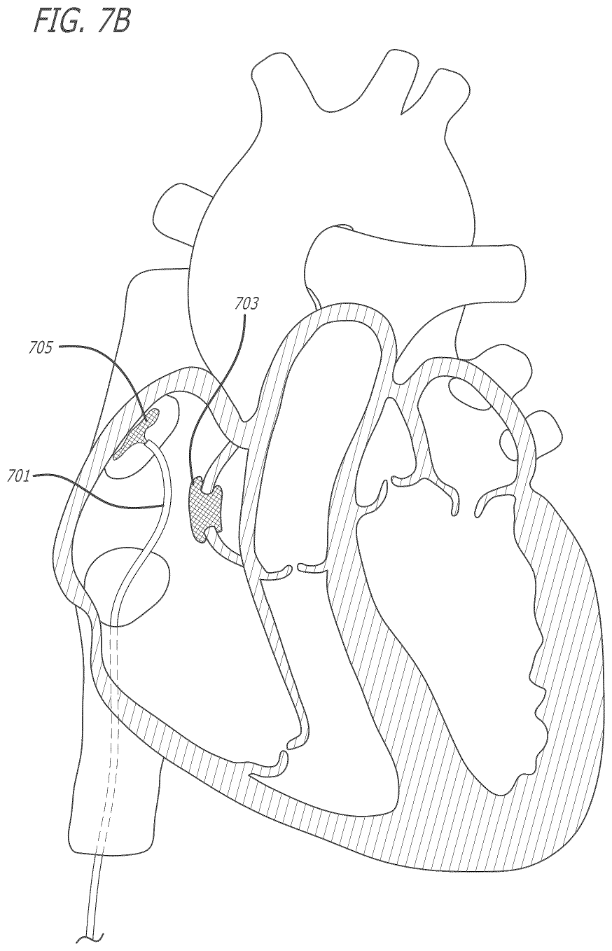

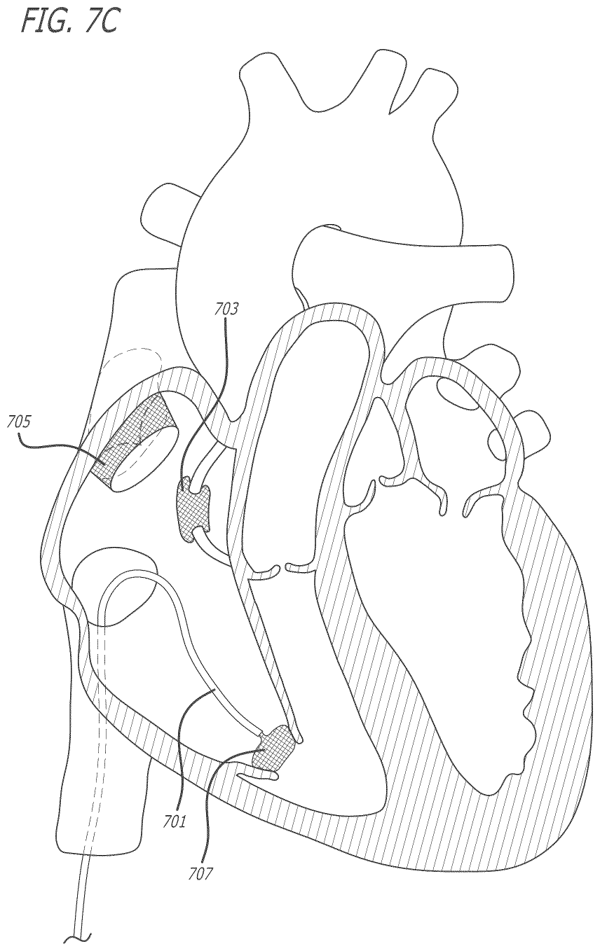

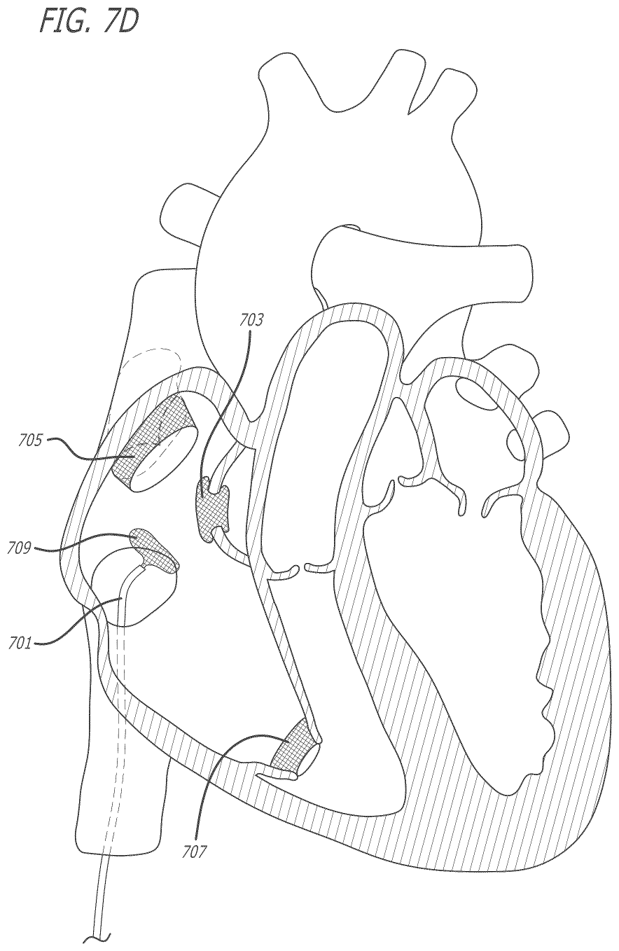

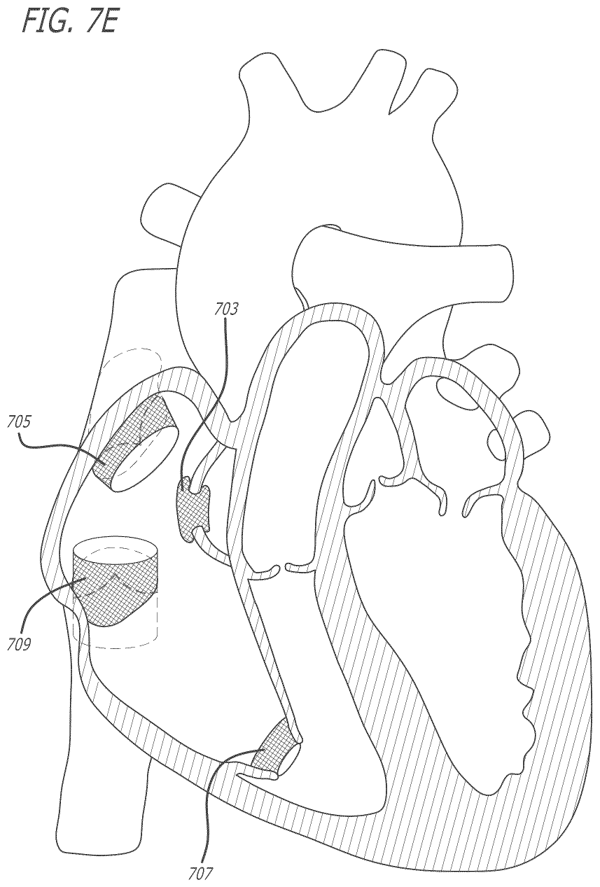

Provided in FIGS. 7A to 7E is an example of a catheter system for installing a set of implants for treatment of Ebstein's anomaly. The system comprises a catheter 701, which can be delivered to the right atrium of the heart (FIG. 7A). Catheter 701 is shown being delivered via the inferior vena cava (e.g., a transfemoral approach) but delivery via the superior vena cava (e.g., transjugular approach) can also be utilized.

At FIG. 7A, a plug 703 is delivered to repair an atrial septal defect. Catheter 701 can be advanced through the atrial septal defect into the left atrium where plug 703 can be expanded therein. Plug 703 is installed on and within the atrial septal defect to occlude the aperture between the two atriums.

At FIG. 7B, a replacement valve 705 is delivered to replace the function of the tricuspid valve. Catheter 701 can be retracted from the atrial septal defect and can be advanced to the junction between the superior vena cava and the right atrium where replacement valve 705 can be expanded therein. Replacement valve 705 is installed on and within the superior vena cava to provide a functional valve and prevent regurgitation back into the superior vena cava. In the alternative, if the approach was via the superior vena cava, a replacement valve 705 is installed on and within the inferior vena cava instead.

At FIG. 7C, a stent 707 is delivered to open up the malformed native tricuspid valve. Catheter 701 can be retracted from the junction between the superior vena cava and can be advanced to the native tricuspid valve where stent 707 can be expanded therein. Stent 707 is installed on and within the native tricuspid valve to open up the valve and pin it to the walls of the left ventricle such that the valve remains open. By keeping the valve open and pinned to the wall, the valve will not disturb blood flow within the left ventricular′ area.

At FIG. 7D, a replacement valve 709 is delivered to replace the function of the tricuspid valve. Catheter 701 can be retracted from the native tricuspid valve back to the junction between the inferior vena cava and right atrium where replacement valve 709 can be expanded therein. Replacement valve 709 is installed on and within the inferior vena cava to provide a functional valve and prevent regurgitation back into the inferior vena cava. In the alternative, if the approach was via the superior vena cava, a replacement valve 709 is installed on and within the superior vena cava instead.

FIG. 7E depicts the result of installation of plug 703, replacement valve 705, stent 707, and replacement valve 709. Effectively, stent 707 prevents the malformed native tricuspid valve from functioning and the combination of replacement valve 705 and replacement valve 709 provides the tricuspid valve function. By this repair, the entirety of the right side the heart (i.e., the right atrium and right ventricle) act as the right ventricle. The superior vena cava and the inferior vena cava act as the right atrium. And the occlusion of the atrial septal defect prevents improper blood flow between the right and left side of the heart.

While a specific configuration of a delivery catheter for delivery of a set of implants is described above with reference to FIG. 7A to 7E, it should be readily appreciated that various configurations of the system can be implemented in any of a variety of combinations of components. Accordingly, the specific configuration of the system described herein should be understood as not to be limited to any specific configuration, but instead can be implemented in any configuration capable of installing the set of implants via a delivery catheter. Likewise, the various steps of the method described above with reference to FIGS. 7A to 7E can all be included and/or some may be omitted, and tailored to the malformations present in the recipient. And additional steps not described here are also possible, which may be necessary to treat the malformations present in the recipient. For example, for the treatment of Ebstein's anomaly, an atrial septal defect is not always present and thus there is no reason to install a plug in the recipient in those situations. Any of the steps described can be performed in any order as well, although the procedure may be easier to perform if any particular install would hinder the installation of other implants. For instance, when using an approach via the inferior vena cava, it is possible to install a replacement valve within the inferior vena cava first, but doing so would hinder the installation of the other implants as the delivery catheter would need to traverse through the installed replacement valve within the inferior vena cava.

1. A system for improving function of a defective heart of a patient, the system comprising:
a tubular membrane encompassing:
a first chamber of the system, shaped to conform to an interior region of a right atrium of the heart, and
a second chamber of the system, shaped to conform to an interior region of a right ventricle of the heart;
a replacement valve, configured to separate the first chamber from the second chamber, and having leaflets for providing flow in only one direction;
a set of one or more inlets for connecting the first chamber with a first portion of cardiovascular anatomy of the patient; and
a set of one or more outlets for connecting the second chamber with a second portion of cardiovascular anatomy of the patient.

2. The system according to claim 1, wherein the system is an integrated system in which the replacement valve is integrated with the tubular membrane, the integrated system being configured to be transcatheterally implanted with the replacement valve integrated with the tubular membrane.

3. The system according to claim 1, wherein the system is a modular system further comprising a dock provided within the membrane, the membrane being configured to be transcatheterally implanted prior to the replacement valve, and the replacement valve being shaped for attachment to the dock within the heart.

4. The system according to claim 1, wherein the membrane comprises a structural layer disposed between two impermeable layers.

5. The system according to claim 4, wherein the structural layer includes an expandable wire mesh frame comprising a shape memory material.

6. The system according to claim 4, wherein the structural layer comprises an expandable wire mesh interconnected to a strut frame.

7. The system according to claim 4, wherein each impermeable layer comprises a stretchable material.

8. The system according to claim 4, wherein each impermeable layer membrane comprises crinkled or folded portions.

9. The system according to claim 4, wherein one of the impermeable layers is an outer layer that comprises a thick fuzzy material.

10. The system according to claim 4, wherein one of the impermeable layers is an outer layer that has a reendothelialization-inducing biologic coating.

11. The system according to claim 1, wherein the membrane is configured to be secured within the cardiovascular anatomy via sutures.

12. The system according to claim 1, wherein the membrane is configured to be secured within the cardiovascular anatomy via stents.

13. Apparatus for treating a patient having Ebstein's anomaly, the apparatus comprising a device that comprises:
a flexible hollow structure for installation in a right side of a heart of the patient, the hollow structure configured to generally conform to interior regions of an atrium and a ventricle of the right side of the heart, wherein blood passes through the hollow structure from an inlet end to an outlet end and wherein the hollow structure includes a one-way valve for replacing a native function of a native tricuspid valve.

14. The apparatus according to claim 13, wherein the hollow structure maintains conformation to a shape of the heart while the heart is beating.

15. The apparatus according to claim 13, further comprising anchors for securing the inlet end and the outlet end of the hollow structure to the heart.

16. The apparatus according to claim 15, wherein the anchors comprise stents for securing the inlet and outlet ends of the hollow structure to the heart.

17. The apparatus according to claim 13, wherein the inlet end is configured to be secured to both an inferior vena cava of the patient and a superior vena cava of the patient, and wherein the outlet end is configured to be secured to a right ventricular outflow tract of the patient, such that the hollow structure provides a replacement conduit extending from the inferior and superior vena cava to the right ventricular outflow tract.

18. The apparatus according to claim 13, wherein the hollow structure is reinforced by a metallic mesh structure.

19. The apparatus according to claim 13, wherein the hollow structure is flexible and stretchable for replacing the function of the right side of the heart.

20. The apparatus according to claim 13, wherein the hollow structure has impermeable walls.
